(19) United States
(12) Patent Application Publication
(10) Pub. No.: US 2020/0330357 A1
(43) Pub. Date: Oct. 22, 2020

(54) TOPICAL DELIVERY SYSTEM CONTAINING CELLULOSE NANOFIBERS

(71) Applicant: ELC MANAGEMENT LLC, Melville, NY (US)

(72) Inventors: Daniela BRATESCU, Northport, NY (US); Snehal M. SHAH, Nesconset, NY (US); Isaac David COHEN, Brooklyn, NY (US); Joe H. COLAS, West Babylon, NY (US); Zhihan ZHOU, Metuchen, NJ (US); Peter A. LANDA, Springfield, NJ (US); Mary Ann SMAIL, Commack, NY (US)

(21) Appl. No.: 16/854,944

(22) Filed: Apr. 22, 2020

Related U.S. Application Data
(60) Provisional application No. 62/836,926, filed on Apr. 22, 2019.

(51) Int. Cl. A61K 8/73; A61Q 19/08; A61Q 19/00; A61K 8/02; A61K 8/67; A61K 8/49; A61K 8/35; A61K 8/34

(52) U.S. Cl. CPC A61K 8/731; A61Q 19/08; A61Q 19/007; A61Q 19/005; A61K 8/027; A61K 8/676; A61Q 17/04; A61K 8/671; A61K 8/35; A61K 8/34; A61K 2800/10; A61K 2800/48; A61K 2800/524; A61K 8/4926

(57) ABSTRACT

Topical delivery systems are described. The topical delivery systems include at least one skin care active and nanocellulose in a cosmetic composition. It has been surprisingly found that the presence of nanocellulose in a cosmetic composition enhances or regulates the penetration of skin care actives. The present application further describes a cosmetic composition comprising a skin care active and nanocellulose and use thereof.

### APPLICATION PRIORITY INFORMATION

This application claims priority to U.S. Provisional Patent Application No. 62/836,926 filed Apr. 22, 2019, the contents of which are incorporated herein by reference in its entirety.

### FIELD

The present disclosure relates generally to cosmetic delivery systems and compositions containing cellulose nanofibers for topical delivery of skin care actives to human skin for cosmetic or dermatological purposes.

### BACKGROUND

Skin has two major layers, namely the epidermis and the dermis. The epidermis is the outer most layer of skin. The dermis is the lower layer of skin that contains collagen and elastin fibers that provide strength to skin and where skin vasculature and nerves are found. The epidermis is composed of five layers. The outermost layer is the stratum corneum, followed by the stratum lucidum, stratum granulosum, stratum spinosum, and, finally the stratum basale as the deepest layer next to the dermis.

Various skin care actives in cosmetic compositions would have to penetrate the stratum corneum. Use of penetration enhancers in topically applied compositions for delivering actives is known. However, there remains a need for improved topical delivery systems for skin care actives and compositions.

### BRIEF DESCRIPTION OF THE DRAWINGS

FIG. 1 shows a comparison of the penetration efficacy of caffeine in a formulation with and without cellulose nanofibers.

FIG. 2 shows a comparison of the penetration efficacy of ascorbic acid 2-glucoside (AA2G) in a formulation with and without cellulose nanofibers.

### SUMMARY

The present invention is directed to a topical delivery system comprising at least one skin care active and nanocellulose in a cosmetic composition, a composition comprising such actives and use thereof, specifically providing a cosmetic benefit, such as skin care, make-up or a combination thereof. The present invention is further directed to a method of modulating the delivery, especially delivery and efficacy of the skin care active in the compositions disclosed herein.

Particularly, the present invention is directed to a topical delivery system comprising at least one skin care active and nanocellulose in a cosmetic composition. The nanocellulose is present in amounts of about 0.001% to about 50% by the total weight of the composition and the skin care active is a negative logP active or a positive logP active. The negative logP active comprises one or more of: niacinamide, kojic acid, 3-o-ethyl-L-ascorbic acid, zincidone (zinc salt of the L-pyrrolidone carboxylic acid), glucosamine, allantoin (5-ureidohydantoin, glyoxylic(acid) diureide), ascorbic acid 2-glucoside, ascorbic acid, lactic acid, glycolic acid, aspartic acid, threonine, glycine, L-ornithine, alanine, lysine, histidine, cysteine, valine, methionine, tyrosine, isoleucine, leucine, phenylalanine, tryptophan, hyaluronic acid, or sodium hyaluronate and the positive logP active comprises one or more of: caffeine, retinol, retinol derivatives, cholesterol, sterols, vitamin E and derivatives, benzophenone and chemical sunscreens, or salicylic acid. Further, the cosmetic composition in the topical delivery system may be one or more of a DNA repair enzyme, a sunscreen active, a humectant, a botanical extract, a peptide, an oil, a thickener, a surfactant, a vitamin, an antioxidant, a preservative, or a dermatologically acceptable carrier and the nanocellulose present in the composition comprises at least one of cellulose nanofibers, cellulose nanocrystals, or bacterial nanocellulose, preferably is phosphate-esterified cellulose nanofibers or crystalline nanocellulose.

The present invention is further directed to a method for regulating or improving topical delivery of a skin care active within the skin, the method comprising topically applying a composition comprising the skin care active and nanocellulose on the skin. The skin care active comprises one or more of niacinamide, kojic acid, 3-o-ethyl-L-ascorbic acid, zincidone (zinc salt of the L-pyrrolidone carboxylic acid), glucosamine, allantoin (5-ureidohydantoin, glyoxylic(acid) diureide), ascorbic acid 2-glucoside, ascorbic acid, lactic acid, glycolic acid, aspartic acid, threonine, glycine, L-ornithine, alanine, lysine, histidine, cysteine, valine, methionine, tyrosine, isoleucine, leucine, phenylalanine, tryptophan, hyaluronic acid, or sodium hyaluronate or one or more of: one or more of caffeine, retinol or retinol derivatives, cholesterol, sterols, vitamin E and derivatives, benzophenone and chemical sunscreens, or salicylic acid. The nanocellulose may comprise at least one of: cellulose nanofibers, cellulose nanocrystals, or bacterial nanocellulose.

A cosmetic or dermatologic composition comprising a skin care active, nanocellulose and cosmetically acceptable carriers is also disclosed such that nanocellulose is present in amounts of about 0.001% to about 50% by the total weight of the composition and the skin care active is a negative logP active or a positive logP active and the composition is substantially free of parabens. The present invention is further directed to a method of treating appearance of human skin by administering to the skin an effective amount of the composition and the treatment provides at least one cosmetic benefit selected from treating/preventing wrinkling, sagging, dry, aged and/or photodamaged skin, boosting, maintaining collagen levels in skin, boosting, maintaining decorin levels in skin, improving skin texture, smoothness or firmness and soothing irritated, red or sensitive skin.

### DESCRIPTION OF THE INVENTION

To facilitate an understanding of this invention, several terms are defined below. Terms defined herein have meanings as commonly understood by a person of ordinary skill in the areas relevant to the present invention. Terms such as “a”, “an” and “the” are not intended to refer to only a singular entity but include the general class of which a specific example may be used for illustration. The terminology herein is used to describe specific embodiments of the invention, but their usage does not delimit the invention, except as outlined in the claims.

The terms “active ingredient” or “active agent” or “cosmetic agent” means a cosmetic agent that is utilized to deliver a benefit to the skin. “Active ingredient” or “active agent” or “cosmetic agent” would cause to, drive a change in the subject's skin or deliver the benefits under consideration, thus, aid in accomplishing a desired, expected, or an intended result. The terms “active ingredient” or “active agent” or “cosmetic agent” according to the present invention include cosmetically acceptable excipients or carriers that may be present in a composition/formulation.

The terms “prevent” and “preventing” include the prevention of the recurrence, spread or onset of a skin or hair condition. It is not intended that the present invention be limited to complete prevention.

The term “subject” refers to any mammal, preferably a human.

The term “topical” refers to administration of an agent or agents (e.g., cosmetic, vitamin, etc.) on the skin.

The terms “transdermal” or “topical” refers to the delivery of an agent (e.g., cosmetic, dermatological, vitamin, etc.) through the skin (e.g., so that at least some portion of the population of particles reaches underlying layers of the skin).

The term “hydrophilic” refers to the physical property of a molecule that is able to transiently associate with water, i.e., bond with water via hydrogen bonding. The term “hydrophobic” or “lipophilic” refers to the physical property of a molecule that is repelled from a mass of water. The term “hydrophobic” is used interchangeably with lipophilic, i.e., “fat-loving”. However, the two terms are not synonymous. While hydrophobic substances are usually lipophilic, there are exceptions, such as the silicones and fluorocarbons.

The term “solvent” refers to a liquid, solid or a gaseous solute generating a solution.

The terms “inhibiting,” “reducing,” or “prevention,” or any variation of these terms, when used in the claims and/or the specification includes any measurable decrease or complete inhibition to achieve a desired result.

The term “effective,” as that term is used in the specification and/or claims, means adequate to accomplish a desired, expected, or intended result.

The term nanofiber refers to fibers having width of about 0.1 to 1000 nanometers.

The term nanofiber refers to fibers having length of greater than 100 nanometers.

The term “logP” value is a constant defined as logP=log10 (Partition Coefficient). LogP algorithms calculate partitioning between octan-1-ol and water, as used in the most common systems.

The term “negative logP” refers to a compound that has a higher affinity for the aqueous phase, therefore it is hydrophilic.

The term “positive logP” refers to a compound that is hydrophobic or lipophilic and denotes a higher concentration of lipid phase.

As referred herein, when logP is zero, such a compound is equally portioned between lipid and aqueous phase.

Except in operating and comparative examples, or where otherwise explicitly indicated, all numbers in this description indicating amounts or ratios of material or conditions of reaction, physical properties of materials and/or use are to be understood as modified by the word “about”. All amounts are presented as percentages by weight of the final cosmetic agent, unless otherwise specified.

The present invention relates to methods for repairing skin using nanocellulose fibers. In some embodiments, the present invention relates to compositions for repairing the skin using nanocellulose present in the cosmetic compositions.

Measurable changes appear in skin as the skin ages or endures environmental or age-related insult. Such insults cause a general reduction in cellular and tissue vitality, reduction in cell replication rates, reduced cutaneous blood flow, reduced moisture content, errors in structure and function, alterations in biochemical pathways, and reduction of skin's ability to remodel and repair itself. It has been surprisingly found that the presence of nanocellulose in a cosmetic or dermatologic composition enhances penetration of negative logP skin care actives within the skin, thereby repairing, treating the appearance of the skin. It has also been surprisingly found that the presence of nanocellulose in a cosmetic composition limits and/or regulates the penetration of positive logP skin care actives, thereby reducing the delivery of the active within the skin.

Nanocellulose refers to nano-structured cellulose. Nanocellulose includes, without limiting, cellulose nanofibers (CNF) (also known as microfibrillated cellulose (MFC)), cellulose nanocrystal (CNC or NCC), or bacterial nanocellulose (nano-structured cellulose produced by bacteria). The nanocellulose fibers typically have a width in the range of 1 nm to 1000 nm, preferably within the range of 1 about nm to about 100 nm and a length of greater than one micrometer, and in some embodiments, the length is greater than 500 nm.

One aspect of the present invention is a topical delivery system. In one embodiment, the topical delivery system comprises at least one skin care active and nanocellulose in a cosmetic composition. Any type of skin care active may be utilized. The skin care actives can be hydrophobic, lipophilic (positive logP active), or hydrophilic (negative logP active). The skin care active may be a small molecule, lipid, peptide, DNA molecules, biomolecules, enzymes or a combination thereof.

According to one embodiment, the skin care actives are negative logP actives. Negative logP actives include, but are not limited to, niacinamide, kojic acid, 3-o-ethyl-L-ascorbic acid, zincidone (zinc salt of the L-pyrrolidone carboxylic acid), glucosamine, allantoin (5-ureidohydantoin, glyoxylic(acid) diureide), ascorbic acid 2-glucoside, ascorbic acid, lactic acid, glycolic acid, aspartic acid, threonine, glycine, L-ornithine, alanine, lysine, histidine, cysteine, valine, methionine, tyrosine, isoleucine, leucine, phenylalanine, tryptophan, hyaluronic acid, sodium hyaluronate, or combinations thereof. According to another embodiment, the skin care active may be positive logP actives, including, without limiting, retinol and retinol derivatives, caffeine, phenoxyethanol, cholesterol, sterols, vitamin E and derivatives, benzophenone, chemical sunscreens, salycilic acid, benzophenone and chemical sunscreens, among others.

According to an embodiment, the cosmetic composition may comprise, but is not limited to, one or more of a DNA repair enzyme, a sunscreen active, a humectant, a botanical extract, a peptide, an oil, a thickener, a surfactant, a vitamin, an antioxidant, a preservative, or a carrier. The carrier is dermatologically or cosmetically acceptable when present in the composition. The nanocellulose may comprise at least one of cellulose nanofibers, cellulose nanocrystals, or bacterial nanocellulose. In some embodiments, the nanocellulose comprises phosphate-esterified cellulose nanofibers. In some embodiments, the nanocellulose comprises crystalline nanocellulose.

In a preferred embodiment, the skin care active comprises a skin care active and the nanocellulose comprises phosphate-esterified cellulose nanofibers.

The composition comprises nanocellulose in ranges are from about 0.0001 to 50%, preferably from about 0.0005 to 40%, more preferably from about 0.001 to 35% of the total weight of the composition.

In another aspect of the present invention is a method of modulating the delivery of skin care active present in a cosmetic composition by utilizing nanocellulose disclosed herein in the composition. The composition according to the present invention comprising nanocellulose and at least one skin care active, wherein the skin care active is a negative p log active and provides enhanced delivery of the active, particularly about 2 to 10 times enhanced efficacious delivery when compared to a composition without nanocellulose. For example, as disclosed in the experiments described herein, without limiting, AA2G when present in a composition according to the present invention provides at least about 3-5 times enhanced delivery of the AA2G when compared to the composition without nanocellulose.

In another embodiment, the composition according to the present invention comprising nanocellulose and at least one skin care active, wherein the skin care active is a positive p log active and it regulates or limits delivery of the active when compared to the composition without nanocellulose. For example, without limiting, positive p log active is selected from a group comprising caffeine, benzophenone and chemical sunscreens, salicylic acid, retinol and derivatives, vitamin e and derivatives, cholesterol, phenoxyethanol, among others.

Delivery of the retinol must be regulated because increased delivery of retinol within the skin might cause irritation to the skin. In one embodiment, retinol when present in a composition according to the present invention limits or reduces the delivery of the retinol within the skin. Retinol is utilized in about 1-4% w/w of the composition. In embodiments, according to the invention, a composition comprising nanocellulose and retinol will regulate the delivery of retinol within the skin, providing for a slow release and reduced delivery within the skin, thereby, avoiding the skin irritation that may be caused to increased delivery of retinol.

In other embodiments, phenoxyethanol is utilized in about 0.1-2% w/w of the composition. In embodiments, according to the invention, a composition comprising nanocellulose and phenoxy ethanol will regulate the delivery within the skin, providing for reduced penetration within the skin.

In other embodiments, chemical sunscreens (benzophenone) is utilized in about 1-6% w/w of the composition. In embodiments, according to the invention, a composition comprising nanocellulose and chemical sunscreens (benzophenone) will regulate the delivery within the skin, providing for reduced penetration within the skin.

In one embodiment, the method of modulating delivery of the skin care active is disclosed wherein the skin care active with positive logP when utilized in a composition (that comprises nanocellulose) limits or regulates delivery of the skin care active within the skin. Accordingly, in one embodiment, the composition disclosed herein comprising nanocellulose may be utilized to modulate, regulate and thereby limit the delivery of the active within the skin that is a positive logP active such as retinol upon topical application. It must be noted that delivery of retinol generally, for example, must be limited because high amounts of retinol may cause irritation to the layers of the skin.

The nanocellulose may comprise at least one of cellulose nanofibers, cellulose nanocrystals, or bacterial nanocellulose. In some embodiments, the nanocellulose comprises phosphate-esterified cellulose nanofibers. In some embodiments, the nanocellulose comprises crystalline nanocellulose.

In a preferred embodiment, the nanocellulose comprises phosphate-esterified cellulose nanofibers. The composition comprises nanocellulose in ranges are from about 0.0001 to 50%, preferably from about 0.0005 to 40%, more preferably from about 0.001 to 35% of the total weight of the composition.

A preferred embodiment of the invention is a topical delivery system to regulate, improve delivery of the active within the skin. In one embodiment, the topical delivery system comprises at least one negative logP skin care active and phosphate-esterified nanocellulose in a cosmetic composition and cosmetically acceptable carriers or softeners.

Another preferred embodiment of the invention is a topical delivery system to regulate, modulate, limit the delivery of the active within the skin. In one embodiment, the topical delivery system comprises at least one positive logP skin care active and phosphate-esterified nanocellulose in a cosmetic composition and cosmetically acceptable carriers or softeners.

In one embodiment, the negative logP skin care active comprises charge/value within the range of −0.01 to about −100. In another embodiment, the positive logP skin care active comprises logP value within the range of about 0.01 to about 100.

Table 1 below, without limiting, shows the logP values of the actives utilized according to the present invention disclosed herein.

TABLE 1

| | |
|---|---|
| niacinamide | −0.37 |
| kojic acid | −0.64 |
| zincidone (zinc salt of the L- | −2.3 |
| pyrrolidone carboxylic acid) | |
| glucosamine | −2.7 |
| allantoin (5-ureidohydantoin, | −2 |
| glyoxylic (acid) diureide) | |
| Ascorbyl glucoside | −2.7 |
| (ascorbic acid 2-glucoside, | |
| AA2G) | |
| ascorbic acid | −1.6 |
| lactic acid | −0.79 |
| glycolic acid | −1.11 |
| aspartic acid | −2.93 |
| threonine | −3.48 |
| glycine | −3.22 |
| L-ornithine | −3.54 |
| alanine | −2.75 |
| lysine | −3.19 |
| histidine | −3.27 |
| cysteine | −2.75 |
| valine | −2.08 |
| methionine | −2.10 |
| tyrosine | −1.51 |
| isoleucine | −1.73 |
| leucine | −1.73 |
| phenylalanine | −1.12 |
| tryptophan | −1.34 |
| hyaluronic acid | −1.8 |
| Caffeine | −0.07 |
| Retinol | 5.68 |
| Retinol palmitate | 10.12 |
| cholesterol | 7.02 |
| Vitamin E (alpha tocopherol) | 8.84 |
| Benzophenone 3 | 3.03 |
| Salycilic acid | 2.26 |
| Avobenzone | 4.98 |
| octinoxate | 5.62 |
| octisalate | 4.79 |
| octocrylene | 6.78 |
| phenoxyethanol | 1.22 |

The nanocellulose may comprise at least one of cellulose nanofibers, cellulose nanocrystals, or bacterial nanocellulose. In some embodiments, the nanocellulose comprises phosphate-esterified cellulose nanofibers. In some embodiments, the nanocellulose comprises crystalline nanocellulose.

In a preferred embodiment, the nanocellulose comprises phosphate-esterified cellulose nanofibers. The composition comprises nanocellulose in ranges are from about 0.0001 to 50%, preferably from about 0.0005 to 40%, more preferably from about 0.001 to 35% of the total weight of the composition.

The composition of the present invention may be formulated as a cosmetic product. In some embodiments, the composition can be formulated to have a pH in a range of about 1-10. In some embodiments, the compositions can be formulated to have a pH of about less than 3.0, 3.5, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, to about 12.0, or more, or any range or integer derivable therein.

The composition can be formulated as a cosmetic product in a cosmetic carrier as an emulsion, cream, lotion, gel, serum, solution, base, spray or foam, among others. In preferred embodiments, the composition is a liquid composition.

In one embodiment, the present invention contemplates a liquid, semi-solid or a solid composition comprising nanocellulose for use on the skin. According to an embodiment, the composition further comprises active ingredient, cosmetic agents or cosmetically acceptable carriers or excipients or softeners.

### Use and Benefits

The composition can be formulated as a cosmetic product in a cosmetically acceptable carrier in form of an emulsion, cream, lotion, gel, serum, solution, spray, base or foam among others for use on the skin. The various skin care actives, nanocellulose, and cosmetic composition components discussed herein can be used in this topical delivery system as well. Such a system may further include a treatment regimen, instructions regarding how to use the composition of the present invention with any other similar cosmetic or make up compositions, treatment regimens or applications.

Another aspect of the present invention is the use of the compositions comprising nanocellulose for personal, cosmetic use, including cosmetics and dermatologically application in human body, (i.e., skin). In some embodiments, the composition may be utilized as a skin care agent or a make-up agent. In some other embodiments, the composition may be utilized as an agent to treat or prevent conditions on the skin as a cosmetic. In some embodiments, the composition may be utilized as a cleansing agent, exfoliating agent or a skin repairing agent. In some embodiments, the composition may be utilized as a makeup or a combination of skin care and makeup product.

Applicants of the present invention unexpectedly and surprisingly achieved the benefit of improving, treating the skin's appearance by using the composition disclosed herein. Specifically, applicants surprisingly discovered that an enhanced delivery of active with a negative p log was achieved when cellulose nanofibers were present in the composition and that delivery of logP positive active was regulated, limited with the cellulose nanofibers in the composition. Applicants also discovered significant benefits, showing a reduction in dilation of the veins, bags under the eyes, dark circles under the eyes, and swelling around the eyes, fine lines and wrinkles, loss of elasticity, wrinkles, loss of stiffness, loss of uniformity of color or tone, rough surface texture, age spots, and a decrease in moisture content in the skin around the eyes and improvement in tightening of the facial skin.

The composition of the present invention may be utilized with other modes of delivery including microneedles, iontophoresis, and/or electroporation. For example, in one embodiment, microneedles are applied to the skin and the composition is applied thereafter. Any and all combinations and permutations in the use is contemplated to be part of the present invention.

In some embodiments, the composition may be formulated for use more than one, two, three, four times a day. In preferred embodiments, the composition may be formulated for use twice a day. In more preferred embodiments, the composition may be formulated for use in the morning and prior to retiring to bed at night.

In particular, the composition disclosed herein may be used to prevent or treat changes to the skin, such as dilation of the veins, bags under the eyes, dark circles under the eyes, and swelling around the eyes, fine lines and wrinkles, loss of elasticity, wrinkles, loss of stiffness, loss of uniformity of color or tone, rough surface texture, age spots, and a decrease in moisture content in the skin. Particularly, the present invention relates to methods for repairing or reducing dilation of the veins, bags under the eyes, dark circles under the eyes, swelling around the eyes, fine lines and wrinkles, loss of elasticity, wrinkles, loss of stiffness, tightening, firming, loss of uniformity of color or tone, rough surface texture, age spots, and a decrease in moisture content in the skin around the eyes.

In additional embodiments, the subject exhibits symptoms associated with or is suspected of having a affected visual appearance, physical properties or physiological functions of the skin, such as visually undesirable appearance to the skin, including dilation of the veins, bags under the eyes, dark circles under the eyes, and swelling around the eyes may occur, environmental damage to the skin, fine lines and wrinkles, loss of elasticity, wrinkles, loss of stiffness, loss of uniformity of color or tone, rough surface texture, age spots, and a decrease in moisture content. Topical application of the composition can treat or prevent such a skin condition. The effectiveness of the composition can be compared with skin that has not been treated or addressed with a composition of the present invention.

In certain non-limiting embodiments, the skin treatment can be localized to and/or around an area (such as eyes) where the composition is applied to the skin. The skin can be facial, torso, back, neck, ear, pelvic, arms, hands, legs (e.g., ankle, knee, thigh), feet, or buttocks skin. Non-limiting examples of skin conditions that can be treated or prevented with compositions of the present invention include telangiectasia (i.e., spider veins), eye circles (e.g., dark circles under the eye), puffy eyes, pruritus, lentigo, age spots, senile purpura, keratosis, melasma, blotches, wrinkles, fine lines, tightening of facial skin, lightening of skin, nodules, sun damaged skin, acne, or hyperpigmentation. In certain aspects, the skin condition can be caused by exposure to UV light, age, irradiation, chronic sun exposure, environmental pollutants, air pollution, wind, cold, heat, chemicals, disease pathologies, treating/preventing wrinkling, sagging, dry, aged and/or photodamaged skin, boosting, maintaining collagen levels in skin, boosting, maintaining or improving decorin, collagen, elastin levels in skin, improving skin texture, smoothness or firmness and soothing irritated, red or sensitive skin. The skin to be treated can be aged, nutritionally compromised, or environmentally damaged skin. In certain aspects, the composition can be topically applied in an amount effective to increase the stratum corneum turnover rate of the skin, collagen synthesis production of the skin, fat production of the skin, firmness of the skin, or elasticity of the skin. In other aspects, the composition can be topically applied in an amount effective to reduce or inhibit new capillary formation in or near the skin, blood flow to the skin, fluid amount in or near the skin, or melanin production in the skin.

In some exemplary embodiments, the invention relates to a composition comprising nanocellulose and active agents or ingredients.

Also, disclosed are systems or kits that can include a composition of the present invention. In certain non-limiting aspects, the composition is comprised in a container. The container can be a bottle, dispenser, package, etc. The container can be configured to dispense a pre-determined amount of the composition. The container can be configured to dispense the composition in a liquid, spray, emulsion, or an aerosol form. In certain aspects, the system or kit can include indicial on its surface and/or instructions for using the composition.

In other aspects of the present invention, the composition can be used as part of a regimen to treat a skin condition. For instance, the regimen can include applying a composition of the present invention in a first instance as disclosed throughout this specification. The regimen can then include additional applications that are identical, similar, or different than the first instance application. The additional applications can include, for example, a second, third, fourth, fifth, sixth, seventh, eighth, nine, tenth, or more applications with a composition of the present invention and/or whether another method for treating a particular skin condition (e.g., other compositions, etc.). The regimen may also include applying the composition in the morning and/or prior to retiring to bed at night.

### Active Ingredients and Forms

According to an aspect of the present invention, the cosmetic composition may comprise, but is not limited to, one or more of a DNA repair enzyme, a sunscreen active, a humectant, a botanical extract, a peptide, an oil, a thickener, a surfactant, a vitamin, an antioxidant, a preservative, or a carrier. If present suggested ranges are from about 0.0001 to 35%, preferably from about 0.0005 to 20%, more preferably from about 0.001 to 15% of the total weight of the composition. The carrier may be dermatologically or cosmetically acceptable present in the composition.

In some embodiments, the composition is a liquid composition. The composition can be formulated as a cosmetic product in a cosmetic carrier as an emulsion, cream, lotion, gel, serum, solution, base or foam.

According to an embodiment, the formulation comprising the cosmetic agent may be applied to mammalian keratinous tissue, to human skin, face or hair. The formulation comprising the cosmetic agents may be of various forms. For example, some non-limiting examples of such forms include solutions, suspensions, lotions, creams, gels, emulsions, suspension, toners, ointments, cleansing agents, exfoliating agents, liquid shampoos and hair conditioners, pastes, foams, powders, mousses, shaving creams, hydrogels, film-forming products, facial and skin masks, and the like.

The formulation type of the cosmetic agents of the present invention may be of any type, including solution system, soluble system, emulsion system, gel system, powder dispersing system or water-oil two phase system.

The composition may be in the form of an aqueous solution, gel, cream, lotion, emulsion, serum, spray, suspension or an emulsion. The emulsion may be either water in oil or oil in water. The composition may also be anhydrous. The composition may be in the liquid, semi-solid, or solid form.

If the composition is present as an aqueous solution or dispersion, the amount of water present may range from about 0.01-99%, and the amount of dissolved or dispersed solids from about 10 to 99.99% by the total weight of the composition. If the composition of the invention is present in the emulsion form, it may comprise from about 0.1-99% water and from about 0.1-80% oil by the total weight of the composition. If the composition of the present invention is in an anhydrous form, it may contain from about 0.1-99% oil.

In one embodiment, the negative logP skin care active comprises charge/value within the range of −0.01 to about −100. In another embodiment, the positive logP skin care active comprises logP value within the range of about 0.01 to about 100. In preferred embodiments, the negative logP skin care active comprises charge/value within the range of −0.01 to about −10 and the positive logP skin care active comprises logP value within the range of about 0.01 to about 10. In embodiments, the compositions can be formulated to have a skin care active with a logP of about −0.01, −0.05, −0.1, −0.7, −1, −2, −5, −10, −2 −0.1, 0.5, 1, 2, 3, 5, 10 or more, or any range or integer derivable therein.

Conventional cosmetic adjuvants which may be suitable as additives are, for example, co-emulsifiers, fats and waxes, stabilizers, thickeners, biogenic agents, film formers, fragrances, dyes, pearlescent agents, preservatives, pigments, electrolytes (for example magnesium sulphate) and pH regulators. Co-emulsifiers are preferably known W/O and also O/W emulsifiers such as polyglycerol esters, sorbitan esters or partially esterified glycerides. Typical examples of fats are glycerides; as waxes which may be mentioned in combination with hydrophilicized growing inter alia beeswax, paraffin wax or microcrystalline waxes. Metal salts of fatty acids such as magnesium, aluminum and/or zinc stearate can be employed. Suitable thickeners are, for example, crosslinked polyacrylic acids and derivatives thereof, polysaccharides, more especially xanthan gum, guar-guar, agar-agar, alginate and tyloses, carboxymethylcellulose and hydroxyethylcellulose, and also fatty alcohols, monoglycerides and fatty acids, polyacrylates, polyvinyl alcohol and polyvinylpyrrolidone. Customary film formers are, for example, hydrocolloids such as chitosan, microcrystalline chitosan or quaternized chitosan, polyvinylpyrrolidone, vinylpyrrolidone-vinyl acetate copolymers, polymers of the acrylic acid series, quaternary cellulose derivatives and similar compounds. Suitable preservatives are, for example, formaldehyde solution, p-hydroxybenzoate or sorbic acid. Pearlizing agents, for example, such as ethylene glycol distearic esters come coldistearate, but also fatty acids and fatty acid into consideration. The dyes suitable for cosmetic purposes, and authorized substances may be used. Such dyes are normally used in concentrations of 0.001 to 0.1 by the weight %, based on the total mixture.

The compositions of the invention can accordingly be in liquid, paste or solid form, for example as a water-in-oil creams, oil-in-water creams and lotions, aerosol foam creams, gels, oils, grease pencils, dusting powders, sprays or hydroalcoholic lotions. Composition may include any active ingredient or cosmetic agent along with cosmetically acceptable excipients or carriers.

### DNA Repair Enzyme

The compositions may also contain one or more DNA repair enzymes. DNA repair enzymes may be present in a range of an amount from about 0.00001 to about 35%, preferably from about 0.00005 to about 30%, more preferably from about 0.0001 to about 25% of one or more DNA repair enzymes by the total weight of the composition.

DNA repair enzymes as disclosed in U.S. Patent Nos. 5,077,211; 5,190,762; 5,272,079; and 5,296,231, are suitable for use in the compositions described herein and method of the present invention. One example of such a DNA repair enzyme may be purchased from AGI/Dermatics under the trade name Roxisomes®, and has the INCI name Arabidopsis Thaliana extract. It may be present alone or in admixture with lecithin and water. This DNA repair enzyme is known to be effective in repairing 8-oxo-diGuanine base mutation damage.

Another type of DNA repair enzyme that may be used is one that is known to be effective in repairing 06-methyl guanine base mutation damage. It is sold by AGI/Dermatics under the tradename Adasomes®, and has the INCI name Lactobacillus ferment, which may be added to the composition of the invention by itself or in admixture with lecithin and water.

Another type of DNA repair enzyme that may be used is one that is known to be effective in repairing T-T dimers. The enzymes are present in mixtures of biological or botanical materials. Examples of such ingredients are sold by AGI/Dermatics under the tradenames Ultrasomes® or Photosomes®. Ultrasomes® comprises a mixture of Micrococcus lysate (an end product of the controlled lysis of various species of Micrococcus), lecithin, and water. Photosomes® comprises a mixture of plankton extract (which is the extract of marine biomass which includes one or more of the following organisms: thalassoplankton, green micro-algae, diatoms, greenish-blue and nitrogen-fixing seaweed), water, and lecithin.

Another type of DNA repair enzyme may be a component of various inactivated bacterial lysates such as Bifida lysate or Bifida ferment lysate, the latter a lysate from Bifido bacteria which contains the metabolic products and cytoplasmic fractions when Bifido bacteria are cultured, inactivated and then disintegrated. This material has the INCI name Bifida Ferment Lysate.

### Formulation

The composition may be in the form of an aqueous solution, gel, cream, lotion, emulsion, serum, spray, suspension or an emulsion. The emulsion may be either water in oil or oil in water. The composition may also be anhydrous. The composition may be in the liquid, semi-solid, or solid form.

If the composition is present as an aqueous solution or dispersion, the amount of water present may range from about 0.01-99%, and the amount of dissolved or dispersed solids from about 10 to 99.99% by the total weight of the composition.

If the composition of the invention is present in the emulsion form, it may comprise from about 0.1-99% water and from about 0.1-80% oil by the total weight of the composition.

If the composition of the present invention is in an anhydrous form, it may contain from about 0.1-99% oil by the total weight of the composition.

### Sunscreens

The compositions of the present invention may comprise one or more sunscreen actives or sunscreen agents. Examples of suitable sunscreen actives include oil-soluble sunscreens, insoluble sunscreens, and water-soluble sunscreens. Non-limiting examples of suitable oil-soluble sunscreens are disclosed in The Cosmetic, Toiletry, and Fragrance Association's The International Cosmetic Ingredient Dictionary and Handbook, 10th Ed., Gottschalck, T. E. and McEwen, Jr., Eds. (2004), p. 2267 and pp. 2292-93 and include benzophenone-3, bis-ethylhexyloxyphenol methoxyphenyl triazine, butyl methoxydibenzoyl-methane, diethylamino hydroxy-benzoyl hexyl benzoate, drometrizole trisiloxane, ethylhexyl methoxy-cinnamate, ethylhexyl salicylate, ethylhexyl triazone, octocrylene, homosalate, polysilicone-15, and derivatives and mixtures thereof. Non-limiting examples of suitable insoluble sunscreens include methylene bis-benzotriazolyl tetramethylbutyl-phenol, titanium dioxide, zinc cerium oxide, zinc oxide, and derivatives and mixtures thereof. Non-limiting examples of suitable water-soluble sunscreens include phenylbenzimidazole sulfonic acid (PBSA), terephthalylidene dicamphor sulfonic acid, (Mexoryl™ SX), benzophenone-4, benzophenone-5, benzylidene camphor sulfonic acid, cinnamidopropyl-trimonium chloride, methoxycinnamido-propyl ethyldimonium chloride ether, disodium bisethylphenyl triaminotriazine stilbenedisulfonate, disodium distyrylbiphenyl disulfonate, disodium phenyl dibenzimidazole tetrasulfonate, methoxycinnamido-propyl hydroxysultaine, methoxycinnamido-propyl laurdimonium tosylate, PEG-25 PABA (p-aminobenzoic acid), polyquaternium-59, TEA-salicylate, and salts, derivatives and mixtures thereof. All known sunscreen actives are considered to be within the scope of the present invention.

### Humectants

The composition may contain one or more humectants. If present, the humectants may range from about 0.1 to 75%, preferably from about 0.5 to 70%, more preferably from about 0.5 to 40% by the total weight of the composition. Examples of suitable humectants include, without limiting, glycols, sugars, and the like. Suitable glycols are in monomeric or polymeric form and include polyethylene and polypropylene glycols such as PEG 4-10, which are polyethylene glycols having from 4 to 10 repeating ethylene oxide units; as well as C₁₋₆ alkylene glycols such as propylene glycol, butylene glycol, pentylene glycol, and the like. Suitable sugars, some of which are also polyhydric alcohols, are also suitable humectants. Examples of such sugars include glucose, fructose, honey, hydrogenated honey, inositol, maltose, mannitol, maltitol, sorbitol, sucrose, xylitol, xylose, and so on. Also suitable is urea. Preferably, the humectants used in the composition of the invention are C₁₋₆, preferably C₂₋₄ alkylene glycols, most particularly butylene glycol, glycerin, propylene glycol, or hexylene glycol.

### Botanical Extracts

It may be desirable to incorporate one more botanical extract into the composition. If present suggested ranges are from about 0.0001 to 20%, preferably from about 0.0005 to 15%, more preferably from about 0.001 to 10% by the total weight of the composition. Suitable botanical extracts include, without limiting, extracts from plants (herbs, roots, flowers, fruits, seeds) such as flowers, fruits, vegetables, and so on, including yeast ferment extract, Padina Pavonica extract, Thermus Thermophilis ferment extract, Camelina Sativa seed oil, Boswellia Serrata extract, olive extract, Acacia Dealbata extract, Acer Saccharinum (sugar maple), Acidopholus, Acorus, Aesculus, Agaricus, Agave, Agrimonia, algae, aloe, citrus, Brassica, cinnamon, orange, apple, blueberry, cranberry, peach, pear, lemon, lime, pea, seaweed, caffeine, green tea, chamomile, willowbark, mulberry, poppy, and those set forth on pages 1646 through 1660 of the CTFA Cosmetic Ingredient Handbook, Eighth Edition, Volume 2. Further specific examples include, but are not limited to, Glycyrrhiza Glabra, Salix Nigra, Macrocycstis Pyrifera, Pyrus Malus, Saxifraga Sarmentosa, Vitis Vinifera, Morus Nigra, Scutellaria Baicalensis, Anthemis Nobilis, Salvia Sclarea, Prunus Amygdalus, Rosmarinus Officianalis, Sapindus makurossi, Caesalpinia spinosa, Citrus Mediaca Limonum, Panax Ginseng, Siegesbeckia Orientalis, Mangifera Indicia, Fructus Mume, Psidium Guajava, Ascophyllum Nodosum, Centaurium erythrea, Glycine Soja extract, Beta Vulgaris, Haberlea Rhodopensis, Polygonum Cuspidatum, Citrus Aurantium Dulcis, Vitis Vinifera, Selaginella Tamariscina, Humulus Lupulus, Citrus Reticulata Peel, Punica Granatum, Asparagopsis, Curcuma Longa, Menyanthes Trifoliata, Helianthus Annuus, Hordeum Vulgare, Cucumis Sativus, Evernia Prunastri, Evernia Furfuracea, Kola Acuminata, glycyrretinic acid, and mixtures thereof.

### Peptides

It may be desirable to incorporate one or more peptides into the composition. The term “peptide” refers to biomolecules having from about 2 to 20 amino acids connected by peptide bonds. Preferred ranges of the peptide present in the composition is from about 0.001 to 20%, preferably from about 0.005 to 15%, more preferably from about 0.01 to 10% by the total weight of the composition. Preferred are biologically active peptides including those set forth in the C.T.F.A. International Cosmetic Ingredient Dictionary and Handbook, Eleventh Edition, 2006, page 2712. Such peptides include, but are not limited to the CTFA names: Acetyl Hexapeptide-1, 7, 8; Acetyl Pentapeptide-1, 2, 3, or 5; Acetyl Tripeptide-1; Acetyl Dipeptide-1 cetyl ester; Acetyl Glutamyl Heptapeptide-3; Acetyl Glutamyl Hexapeptide-6; Acetyl Monofluoropeptide-1; Heptapeptide-1, 2, or 3; Hexapeptide-1, 2, 3, 4, 5, 6, 7, 8, 9, 10, 11, 12, 13, or 14; Manganese Tripeptide-1; Myristoyl Hexapeptide-5, 12, or 13; Myristoyl Nonapeptide-2; Myristoyl Pentapeptide-4; Myristoyl Tetrapeptide-4 or 6; Myristoyl Tripeptide-4; Nisin, Nonapeptide-1 or 2; Oligopeptide-1, 2, 3, 4, 5, 6, 7, 8, 9 or 10; Palmitoyl Hexapeptide-14; Palmitoyl Pentapeptide-4; Palmitoyl Pentapeptide-4 or 5; Palmitoyl Tripeptide-1 or 5; Pentapeptide-1, 2, 3, 4, 5, or 6; Tetrapeptide-1, 2, 3, 4, 5, 6, or 7; Tripeptide-1, 2, 3, 4, or 5; or Palmitoyl Oligopeptides. All peptides that has cosmetic or dermatologic applications are considered to be within the scope of the present invention.

In one preferred embodiment the composition comprises Acetyl Hexapeptide-8, having the trade name Argireline®.

### Oils

The composition may also comprise one or more oils in the form of natural, synthetic, or silicone oils. The term “oil” refers to an ingredient that is pourable at room temperature, e.g., 25° C. Oils may be volatile or non-volatile. The term “volatile” means that the oil has vapor pressure greater than about 2 mm of mercury at 20° C. The term “non-volatile” means that the oil has a vapor pressure of less than about 2 mm. of mercury at 20° C. If present, suggested ranges are from about 0.1 to 60%, preferably from about 0.5 to 45% by the total weight of the composition.

Examples of volatile oils include volatile linear, cyclic or branched silicones such as cyclopentasiloxane, cyclohexasiloxane (2 cst), hexamethyldisiloxane (0.65 cst, centistokes), octamethyltrisiloxane (1.0 cst), decamethyltetrasiloxane (1.5 cst), or dodecamethylpentasiloxane (2.0 cst); or branched volatile silicones such as methyl trimethicone (1.5 cst). Also suitable are volatile paraffinic hydrocarbons such as isododecane, isohexadecane, C11-14 alkanes, and mixtures thereof.

Non-volatile oils include linear silicones commonly referred to as dimethicone, phenyl substituted silicones such as phenyl dimethicone, phenyl trimethicone, trimethylsiloxy phenyldimethicone, cetyl dimethicone, perfluorodimethicone, phenethyl dimethicone, and the like.

Non-volatile oils may also include esters or hydrocarbons. Esters include C1-10 alkyl esters of C1-20 carboxylic acids. One preferred type of ester is a fatty acid (C6-22) ester of a straight or branched chain saturated or unsaturated C1-22 alkyl. Examples include esters that have a low viscosity, e.g., ranging from 10-100 cst at room temperature. Examples of such esters include but are not limited to jojoba esters. Other non-volatile oils include sterols such as phytosterols, phytosphingosine, and similar plant sterols.

### Thickeners

Suitable thickeners may be incorporated into the composition. Suitable thickeners may be present in ranges are from about 0.0001-45%, preferably from about 0.0005-40% by the total weight of the composition.

Examples of thickeners include animal, vegetable, mineral, silicone, or synthetic waxes which may have melting points ranging from about 30 to 150° C. including, but not limited to waxes made by Fischer-Tropsch synthesis, such as polyethylene or synthetic wax or various vegetable waxes such as bayberry, candelilla, ozokerite, acacia, beeswax, ceresin, cetyl esters, flower wax, citrus wax, carnauba wax, jojoba wax, Japan wax, polyethylene, microcrystalline, rice bran, lanolin wax, mink, montan, bayberry, ouricury, ozokerite, palm kernel wax, paraffin, avocado wax, apple wax, shellac wax, clary wax, spent grain wax, grape wax, and polyalkylene glycol derivatives thereof such as PEG6-20 beeswax, or PEG-12 carnauba wax or fatty acids or fatty alcohols, including esters thereof, such as hydroxystearic acids (for example 12-hydroxy stearic acid), tristearin, tribehenin, and so on.

Also, suitable thickening agents such as silicas, silicates, silica silylate, and alkali metal or alkaline earth metal derivatives thereof may be utilized in the composition. These silicas and silicates are generally found in the particulate form and may include silica, silica silylate, magnesium aluminum silicate, and the like.

Silicone elastomers may also be used as thickening agents. Such elastomers include those formed by addition reaction-curing, by reacting an SiH-containing diorganosiloxane and an organopolysiloxane having terminal olefinic unsaturation, or an alpha-omega diene hydrocarbon, in the presence of a platinum metal catalyst. Such elastomers may also be formed by other reaction methods such as condensation-curing organopolysiloxane compositions in the presence of an organotin compound via a dehydrogenation reaction between hydroxyl-terminated diorganopolysiloxane and SiH-containing diorganopolysiloxane or alpha omega diene or by condensation-curing organopolysiloxane compositions in the presence of an organotin compound or a titanate ester using a condensation reaction between an hydroxyl-terminated diorganopolysiloxane and a hydrolysable organosiloxane; peroxide-curing organopolysiloxane compositions which thermally cure in the presence of an organoperoxide catalyst.

One type of elastomer that may be suitable is prepared by addition reaction-curing an organopolysiloxane having at least 2 lower alkenyl groups in each molecule or an alpha-omega diene; and an organopolysiloxane having at least 2 silicon-bonded hydrogen atoms in each molecule; and a platinum-type catalyst. While the lower alkenyl groups such as vinyl, can be present at any position in the molecule, terminal olefinic unsaturation on one or both molecular terminals is preferred. The molecular structure of this component may be straight chain, branched straight chain, cyclic, or a network. These organopolysiloxanes are exemplified by methylvinylsiloxanes, methylvinylsiloxane-dimethylsiloxane copolymers, dimethylvinylsiloxy-terminated dimethylpolysiloxanes, dimethylvinylsiloxy-terminated dimethylsiloxane-methylphenyl siloxane copolymers, dimethylvinylsiloxy-terminated dimethyl siloxane-diphenyl siloxane-methylvinylsiloxane copolymers, trimethylsiloxy-terminated dimethylsiloxane-methylvinylsiloxane copolymers, trimethylsiloxy-terminated dimethylsiloxane-methylphenylsiloxane-methylvinylsiloxane copolymers, dimethylvinylsiloxy-terminated methyl(3,3,3-trifluoropropyl) polysiloxanes, and dimethylvinylsiloxy-terminated dimethylsiloxane-methyl(3,3,-trifluoropropyl)siloxane copolymers, decadiene, octadiene, heptadiene, hexadiene, pentadiene, or tetradiene, or tridiene.

Curing proceeds by the addition reaction of the silicon-bonded hydrogen atoms in the dimethyl methylhydrogen siloxane, with the siloxane or alpha-omega diene under catalysis using the catalyst mentioned herein. To form a highly crosslinked structure, the methyl hydrogen siloxane must contain at least 2 silicon-bonded hydrogen atoms in each molecule in order to optimize function as a crosslinker.

The catalyst used in the addition reaction of silicon-bonded hydrogen atoms and alkenyl groups, and is concretely exemplified by chloroplatinic acid, possibly dissolved in an alcohol or ketone and this solution optionally aged, chloroplatinic acid-olefin complexes, chloroplatinic acid-alkenylsiloxane complexes, chloroplatinic acid-diketone complexes, platinum black, and carrier-supported platinum.

Examples of suitable silicone elastomers for use in the compositions of the invention may be in the powder form or dispersed or solubilized in solvents such as volatile or non-volatile silicones, or silicone compatible vehicles such as paraffinic hydrocarbons or esters. Examples of silicone elastomer powders include vinyl dimethicone/methicone silesquioxane crosspolymers like Shin-Etsu's KSP-100, KSP-101, KSP-102, KSP-103, KSP-104, KSP-105, hybrid silicone powders that contain a fluoroalkyl group like Shin-Etsu's KSP-200 which is a fluoro-silicone elastomer, and hybrid silicone powders that contain a phenyl group such as Shin-Etsu's KSP-300, which is a phenyl substituted silicone elastomer; and Dow Coming's DC 9506. Examples of silicone elastomer powders dispersed in a silicone compatible vehicle include dimethicone/vinyl dimethicone crosspolymers supplied by a variety of suppliers including Dow Corning Corporation under the tradenames 9040 or 9041, GE Silicones under the tradename SFE 839, or Shin-Etsu Silicones under the trade names KSG-15, 16, 18. KSG-15 has the CTFA name cyclopentasiloxane/dimethicone/vinyl dimethicone crosspolymer. KSG-18 has the INCI name phenyl trimethicone/dimethicone/phenyl vinyl dimethicone crosspolymer. Silicone elastomers may also be purchased from Grant Industries under the Gransil trademark. Also suitable are silicone elastomers having long chain alkyl substitutions such as lauryl dimethicone/vinyl dimethicone crosspolymers supplied by Shin Etsu under the tradenames KSG-31, KSG-32, KSG-41, KSG-42, KSG-43, and KSG-44. Cross-linked organopolysiloxane elastomers useful in the present invention and processes for making them are further described in U.S. Pat. No. 4,970,252 to Sakuta et al., issued Nov. 13, 1990; U.S. Pat. No. 5,760,116 to Kilgour et al., issued Jun. 2, 1998; U.S. Pat. No. 5,654,362 to Schulz, Jr. et al. issued Aug. 5, 1997; and Japanese Patent Application JP 61-18708, assigned to Pola Kasei Kogyo KK.

Polysaccharides may be suitable aqueous phase thickening agents. Examples of such polysaccharides include naturally derived materials such as agar, agarose, alicaligenes polysaccharides, algin, alginic acid, acacia gum, amylopectin, chitin, dextran, cassia gum, cellulose gum, gelatin, gellan gum, hyaluronic acid, hydroxyethyl cellulose, methyl cellulose, ethyl cellulose, pectin, sclerotium gum, xanthan gum, pectin, trehelose, gelatin, and so on.

Also suitable are different types of synthetic polymeric thickeners. One type includes acrylic polymeric thickeners comprised of monomers A and B wherein A is selected from the group consisting of acrylic acid, methacrylic acid, and mixtures thereof; and B is selected from the group consisting of a C1-22 alkyl acrylate, a C1-22 alky methacrylate, and mixtures thereof are suitable. Acrylic polymer solutions include those sold by Seppic, Inc., under the tradename Sepigel® or those sold under the tradename Aristoflex®.

Also suitable are acrylic polymeric thickeners that are copolymer of A, B, and C monomers wherein A and B are as defined above, and C has the general formula:

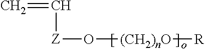

wherein Z is —(CH₂)ₘ; wherein m is 1-10, n is 2-3, o is 2-200, and R is a C₁₀₋₃₀ straight or branched chain alkyl. Examples of the secondary thickening agent above, are copolymers where A and B are defined as above, and C is CO, and wherein n, o, and R are as above defined. Examples of such secondary thickening agents include acrylates/steareth-20 methacrylate copolymer, which is sold by Rohm & Haas under the tradename Acrysol ICS-1.

Also suitable are acrylate based anionic amphiphilic polymers containing at least one hydrophilic unit and at least one allyl ether unit containing a fatty chain. Preferred are those where the hydrophilic unit contains an ethylenically unsaturated anionic monomer, more specifically a vinyl carboxylic acid such as acrylic acid, methacrylic acid or mixtures thereof, and where the allyl ether unit containing a fatty chain corresponds to the monomer of formula:

CH₂=CR′CH₂OBₙR

in which R′ denotes H or CH₃, B denotes the ethylenoxy radical, n is zero or an integer ranging from 1 to 100, R denotes a hydrocarbon radical selected from alkyl, arylalkyl, aryl, alkylaryl and cycloalkyl radicals which contain from 8 to 30 carbon atoms, preferably from 10 to 24, and even more particularly from 12 to 18 carbon atoms. More preferred in this case is where R′ denotes H, n is equal to 10 and R denotes a stearyl (C18) radical. Anionic amphiphilic polymers of this type are described and prepared in U.S. Pat. Nos. 4,677,152 and 4,702,844. Among these anionic amphiphilic polymers, polymers formed of 20 to 60% by weight acrylic acid and/or methacrylic acid, of 5 to 60% by weight lower alkyl methacrylates, of 2 to 50% by weight allyl ether containing a fatty chain as mentioned above, and of 0 to 1% by weight of a crosslinking agent which is a well-known copolymerizable polyethylenic unsaturated monomer, for instance diallyl phthalate, allyl (meth)acrylate, divinylbenzene, (poly)ethylene glycol dimethacrylate and methylenebisacrylamide. One commercial example of such polymers are crosslinked terpolymers of methacrylic acid, of ethyl acrylate, of polyethylene glycol (having 10 EO units) ether of stearyl alcohol or steareth-10, in particular those sold by the company Allied Colloids under the names SALCARE SC80 and SALCARE SC90, which are aqueous emulsions containing 30% of a crosslinked terpolymer of methacrylic acid, of ethyl acrylate and of steareth-10 allyl ether (40/50/10).

Also suitable are acrylate copolymers such as Polyacrylate-3 which is a copolymer of methacrylic acid, methylmethacrylate, methylstyrene isopropylisocyanate, and PEG-40 behenate monomers; Polyacrylate-10 which is a copolymer of sodium acryloyldimethyltaurate, sodium acrylate, acrylamide and vinyl pyrrolidone monomers; or Polyacrylate-11, which is a copolymer of sodium acryloyldimethylacryloyldimethyl taurate, sodium acrylate, hydroxyethyl acrylate, lauryl acrylate, butyl acrylate, and acrylamide monomers.

Also suitable are crosslinked acrylate based polymers where one or more of the acrylic groups may have substituted long chain alkyl (such as 6-40, 10-30, and the like) groups, for example acrylates/C10-30 alkyl acrylate crosspolymer which is a copolymer of C10-30 alkyl acrylate and one or more monomers of acrylic acid, methacrylic acid, or one of their simple esters crosslinked with the allyl ether of sucrose or the allyl ether of pentaerythritol. Such polymers are commonly sold under the Carbopol or Pemulen tradenames and have the CTFA name carbomer.

One particularly suitable type of aqueous phase thickening agent are acrylate based polymeric thickeners sold by Clariant under the Aristoflex trademark such as Aristoflex AVC, which is ammonium acryloyldimethyltaurate/VP copolymer; Aristoflex AVL which is the same polymer has found in AVC dispersed in mixture containing caprylic/capric triglyceride, trilaureth-4, and polyglyceryl-2 sesquiisostearate; or Aristoflex HMB which is ammonium acryloyldimethyltaurate/beheneth-25 methacrylate crosspolymer, and the like. Also, suitable as thickening agents are various polyethylene glycols (PEG) derivatives where the degree of polymerization ranges from 1,000 to 200,000. Such ingredients are indicated by the designation “PEG” followed by the degree of polymerization in thousands, such as PEG-45M, which means PEG having 45,000 repeating ethylene oxide units. Examples of suitable PEG derivatives include PEG 2M, 5M, 7M, 9M, 14M, 20M, 23M, 25M, 45M, 65M, 90M, 115M, 160M, 180M, and the like.

Also suitable are polyglycerins which are repeating glycerin moieties where the number of repeating moieties ranges from 15 to 200, preferably from about 20-100. Examples of suitable polyglycerins include those having the CTFA names polyglycerin-20, polyglycerin-40, and the like.

### Surfactants

The compositions of the invention may contain one or more surfactants. This is particularly desirable when the composition is in the form of an aqueous gel or emulsion. If present, the surfactant may range from about 0.001 to 50%, preferably from about 0.005 to 40%, more preferably from about 0.01 to 35% by weight of the total composition. Suitable surfactants may be silicone or organic, nonionic, anionic, amphoteric or zwitterionic. Such surfactants include, but are not limited to, those set forth herein and are well known in the art.

### Vitamins and Antioxidants

The compositions of the invention may contain vitamins and/or coenzymes, as well as antioxidants. The composition may include vitamins and/coenzymes in amounts ranging from about 0.001-10%, preferably 0.01-8%, more preferably 0.05-5% by weight of the total composition is suggested. Suitable vitamins include ascorbic acid and derivatives thereof such as ascorbyl palmitate, tetrahexydecyl ascorbate, and the B vitamins such as thiamine, riboflavin, pyridoxin, as well as coenzymes such as thiamine pyrophoshate, flavin adenin dinucleotide, folic acid, pyridoxal phosphate, tetrahydrofolic acid, and so on. Also, Vitamin A and derivatives thereof are suitable. Examples are retinyl palmitate, retinol. retinoic acid, as well as Vitamin A in the form of beta carotene. Also, suitable is Vitamin E and derivatives thereof such as Vitamin E acetate, nicotinate, or other esters thereof. In addition, Vitamins D and K are suitable.

Suitable antioxidants are ingredients which assist in preventing or retarding spoilage. Examples of antioxidants suitable for use in the compositions of the invention are potassium sulfite, sodium bisulfite, sodium erythrobate, sodium metabisulfite, sodium sulfite, propyl gallate, cysteine hydrochloride, butylated hydroxytoluene, butylated hydroxyanisole, and so on.

### Preservatives

The composition may contain 0.001-8%, preferably 0.01-6%, more preferably 0.05-5% by weight of the total composition of preservatives. A variety of preservatives are suitable, including such as benzoic acid, benzyl alcohol, benzylhemiformal, benzylparaben, 5-bromo-5-nitro-1,3-dioxane, 2-bromo-2-nitropropane-1,3-diol, butyl paraben, phenoxyethanol, methyl paraben, propyl paraben, diazolidinyl urea, calcium benzoate, calcium propionate, caprylyl glycol, biguanide derivatives, phenoxyethanol, captan, chlorhexidine diacetate, chlorhexidine digluconate, chlorhexidine dihydrochloride, chloroacetamide, chlorobutanol, p-chloro-m-cresol, chlorophene, chlorothymol, chloroxylenol, m-cresol, o-cresol, DEDM Hydantoin, DEDM Hydantoin dilaurate, dehydroacetic acid, diazolidinyl urea, dibromopropamidine diisethionate, DMDM Hydantoin, and the like. In one preferred embodiment the composition is free of parabens.

### Particulate Materials

The compositions of the invention may contain particulate materials in the form of pigments, inert particulates, or mixtures thereof. Such particulate material may be present in ranges from about 0.01-75%, preferably about 0.5-70%, more preferably about 0.1-65% by weight of the total composition. In the case where the composition may comprise mixtures of pigments and powders, suitable ranges include about 0.01-75% pigment and 0.1-75% powder, such weights by weight of the total composition.

A. Powders

The particulate matter may be colored or non-colored (for example, white) non-pigmented powders. Suitable non-pigmented powders include bismuth oxychloride, titanated mica, fumed silica, spherical silica, polymethylmethacrylate, micronized teflon, boron nitride, acrylate copolymers, aluminum silicate, aluminum starch octenylsuccinate, bentonite, calcium silicate, cellulose, chalk, corn starch, diatomaceous earth, fuller's earth, glyceryl starch, hectorite, hydrated silica, kaolin, magnesium aluminum silicate, magnesium tri silicate, maltodextrin, montmorillonite, microcrystalline cellulose, rice starch, silica, talc, mica, titanium dioxide, zinc laurate, zinc myristate, zinc rosinate, alumina, attapulgite, calcium carbonate, calcium silicate, dextran, kaolin, nylon, silica silylate, silk powder, sericite, soy flour, tin oxide, titanium hydroxide, trimagnesium phosphate, walnut shell powder, or mixtures thereof. The above-mentioned powders may be surface treated with lecithin, amino acids, mineral oil, silicone, or various other agents either alone or in combination, which coat the powder surface and render the particles more lipophilic in nature.

B. Pigments

The particulate materials may comprise various organic and/or inorganic pigments. The organic pigments are generally various aromatic types including azo, indigoid, triphenylmethane, anthroquinone, and xanthine dyes which are designated as D&C and FD&C blues, browns, greens, oranges, reds, yellows, etc. Organic pigments generally consist of insoluble metallic salts of certified color additives, referred to as the Lakes. Inorganic pigments include iron oxides, ultramarines, chromium, chromium hydroxide colors, and mixtures thereof Iron oxides of red, blue, yellow, brown, black, and mixtures thereof are suitable.

### Carrier

The compositions comprise a dermatologically or cosmetically acceptable carrier for the skin care active materials. “Dermatologically/cosmetically acceptable,” as used herein, means that the compositions or components described are suitable for use in contact with human keratinous tissue without undue toxicity, incompatibility, instability, allergic response, and the like. The compositions may comprise from about 50% to about 99.99%, alternatively from about 60% to about 99.9%, alternatively from about 70% to about 98%, and alternatively from about 80% to about 95% of the composition by the total weight of the composition. The carrier can be a wide variety of types, non-limiting examples of which include solutions, dispersions, emulsions and combinations thereof. “Emulsions” generally contain an aqueous phase and an oil phase. The oils may be derived from animals, plants, or petroleum, may be natural or synthetic, and may include silicone oils. Emulsion carriers include but are not limited to oil-in-water, water-in-oil and water-in-oil-in-water emulsions. In one embodiment, the carrier comprises an oil-in-water emulsion, a water-in-oil emulsion a silicone-in-water emulsion, and/or a water-in-silicone emulsion. The emulsions may comprise from about 0.01% to about 10%, and alternatively from about 0.1% to about 5%, of a nonionic, anionic or cationic emulsifier, and combinations thereof. Suitable emulsifiers are disclosed in, for example, U.S. Pat. Nos. 3,755,560, 4,421,769, and McCutcheon's Detergents and Emulsifiers, North American Edition, pages 317-324 (1986).

The invention will be further described in connection with the following examples which are set forth for purposes of illustration only.

### EXAMPLES AND EXPERIMENTS

The delivery efficacy of actives with different hydrophilicities in formulations with and without the use of nanocellulose was studied. In particular, the delivery efficacy of caffeine and ascorbic acid 2-glucoside (AA2G) from a formula containing nanocellulose was analyzed.

A testing formulation with and without nanocellulose and various skin care actives was prepared. The testing formulation is shown in Table 2.

TABLE 2

| Component | % |
|---|---|
| Phase 1 | |
| Water | QS 100 |
| Skin Care Active (for example, caffeine, | 0.2 |
| AA2G, etc) | |
| Preservative | 0.05 |
| Phase 2 | |
| Additional Skin Care Active | 0.2 |
| Surfactant | 0.5 |
| Phase 3 | |
| Volatile and Non-volatile Oils/Botanical | 23 |
| Extracts/Silicone Elastomer | |
| Volatile Oils/ Thickener/Silicone Elastomer | 10 |
| Non-volatile Oil | 5 |
| Volatile Oil | 2 |
| Surfactant | 1.5 |
| Phase 4 | |
| Water/Cellulose/Preservative (1.5% cellulose); | 30 |
| Cellulose is phosphate esterified cellulose | |
| nanofibers, commercially available as | |
| Barclear CS) | |
| Humectants/Preservatives | 6.6 |
| Vitamin E [or other actives at its respective | 0.2 |
| % w.w. by the total composition] | |
| Phase 5 | |
| AA2G (ascorbyl glucoside) | 1.8 |
| Water | 4 |
| pH Adjuster | 0.65 |
| Phase 6 | |
| Thickeners/Volatile Oils/Surfactant/Water | 1.5 |

The formulation was prepared by mixing the Phase I components until all of the solids were dissolved (e.g., about 10-15 min). The Phase 2 components were added to the Phase 1 components and mixed until lump free (e.g., about 10 min). The Phase 3 components were pre-mixed until lump free and slowly added to the initial mixture and mixed until uniform (e.g., about 30 min). The Phase 4 components were mixed until uniform and slowly added to the initial mixture and mixed to uniformity (e.g., about 15-20 min). The Phase 5 components were premixed and then added to the mixture and mixed until uniform (e.g., about 15-20 min). The Phase 6 components were added to the mixture under Silverson to adjust the pH to about 5.5 to 6.5 to increase the stability of the AA2G and mixed until lump free (e.g., about 15-20 min).

A penetration study was then performed using the base formula without nanocellulose as a control. The penetration efficacies of caffeine (positive p log active) and AA2G (negative p log active) were then compared.

### Penetration Study Using MatTek Skin.

In a 6-well plate, MatTek 300 skin (a skin model constructed from live cells, obtained from MatTek) was placed in the well with 2.00 mL of incubation medium (also provided by MatTek). The medium was removed, and 2.0 mL of phosphate buffer saline/propandiol (PBS/PPD) 1:1 mixture was added. The PBS/PPD mixture was then removed. This washing process was repeated twice. Then 2.0 mL of the PBS/PPD mixture was added as the collection phase. To the top of each skin, 400 μl of a sample was loaded. The skin was then incubated at 37° C. for 72 hours. Finally, the PBS/PPD collection phase was filtered through 0.45 μm PTFE syringe filter into high performance liquid chromatography (HPLC) vials.

### UPLC Study

Waters ACQUITY H ultra-performance liquid chromatography (UPLC) was used to analyze the collected samples from the penetration study. The UPLC parameters are shown in Table 3 below.

TABLE 3
UPLC study parameters.

| | | Caffeine | AA2G |
|---|---|---|---|
| Column | XSelect HSS T3 2.5 um | | |
| | 3.0 * 50 mm Column XP | | |
| Mobile Phase | 60 % DI water, | | 0.1 % Formic |
| | 40 % of 0.1 % Formic | | Acid in |
| | Acid in Acetonitrile | | DI water |
| Flow rate | 0.80 mL/min | | |
| Column Temp (° C.) | 40 | | |
| Injection volume (ul) | 2 | | |
| Detection wavelength (nm) | 280 | 254 | 254 |

### Example 1

### Penetration of Caffeine

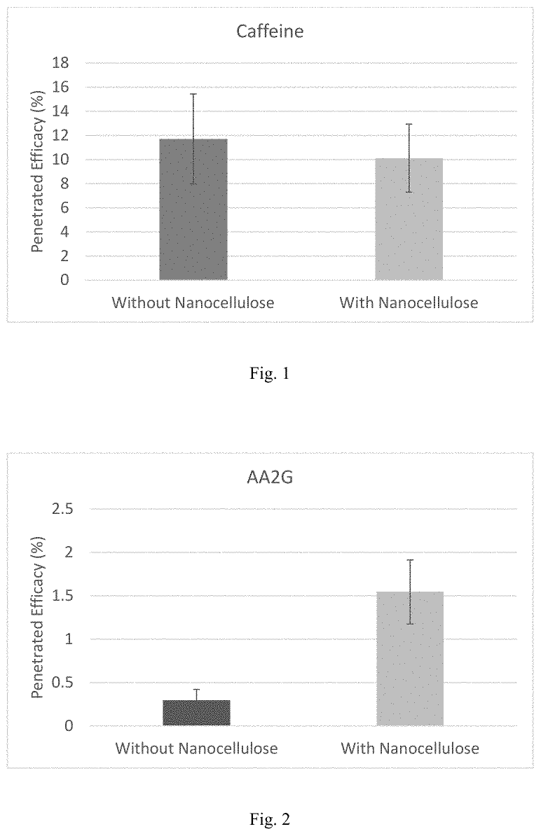

Caffeine, a positive logP active (with a logP of 0.07 and it is equally partitioned between lipid and aqueous phase) is a skin care active that can be dissolved in both water and hydrophobic solvents. The penetration efficacy of caffeine from the two formulations is shown in FIG. 1. The penetration efficacy of caffeine did not show a significant difference between the formulation with cellulose nanofibers and without cellulose nanofibers.

### Example 2

### Penetration of AA2G

AA2G is a negative logP skin care active. It may penetrate through the skin barrier with a relatively lower efficacy than caffeine. The penetration efficacy of AA2G of the two formulations is shown in FIG. 2. The penetration efficacy of AA2G of the formulation with cellulose nanofibers is 5 times greater than from the control formulation without cellulose nanofibers.

The penetration efficacy of AA2G was significantly enhanced by the addition of cellulose nanofibers to the formulation. The penetration efficacy of caffeine was unaffected by the presence of the cellulose nanofibers.

### Example 3

### Penetration of Phenoxyethanol 1% (w.w)

Phenoxyethanol is a positive logP active. From experiments, it is observed that the penetration efficacy of phenoxyethanol 1% (w/w of the total composition according to the present invention) is not increased. When the composition according to the present invention is utilized, penetration of phenoxyethanol is not increased within the skin than the composition without nanocellulose.

### Example 4

### Penetration of Benzophenone 4% (w.w) in Sunscreen Formulations

Benzophenone is a positive logP active. Penetration of benzophenone must be controlled, especially when utilized in sunscreen formulations. From experiments, it is observed that the penetration efficacy of benzophenone 4% (w/w of the total composition according to the present invention) is directionally reduced. When the composition according to the present invention is utilized, penetration of benzophenone is directionally reduced within the skin than the composition without nanocellulose.

### Example 5

### Penetration of Retinol 0.3%

Also, retinol is a positive logP active. Penetration of retinol must be controlled to modulate the release kinetics because increased amount of retinol within the skin causes skin irritation. From experiments, it is observed that the penetration efficacy of retinol 0.3% (w/w of the total composition according to the present invention) is modulated to achieve slower rate of release and thereby, penetration within the skin, avoiding skin irritation. Thus, when the composition according to the present invention is utilized, penetration of retinol is reduced within the skin than the composition without nanocellulose.

In summary, a composition comprising nanocellulose, the penetration efficacy of a negative logP active was significantly enhanced while the penetration efficacy of a logP active such as caffeine and UP302 is modulated, reduced, and limited as shown in the results herein. Further, this is consistent with the previous studies, including experiments with AA2G.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where neither or both limits are included is also encompassed within the invention. Where a value being discussed has inherent limits, for example where a component can be present at a concentration of from 0 to 100%, or where the pH of an aqueous solution can range from 1 to 14, those inherent limits are specifically disclosed. Where a value is explicitly recited, it is to be understood that values which are about the same quantity or amount as the recited value are also within the scope of the invention, as are ranges based thereon. Where a combination is disclosed, each sub combination of the elements of that combination is also specifically disclosed and is within the scope of the invention. Conversely, where different elements or groups of elements are disclosed, combinations thereof are also disclosed. Where any element of an invention is disclosed as having a plurality of alternatives, examples of that invention in which each alternative is excluded singly or in any combination with the other alternatives are also hereby disclosed; more than one element of an invention can have such exclusions, and all combinations of elements having such exclusions are hereby disclosed.

Every document cited herein, including any cross referenced or related patent or application is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A topical delivery system comprising at least one skin care active and nanocellulose in a cosmetic composition, wherein the nanocellulose is present in amounts from about 0.001% to about 50% by the total weight of the composition.

2. The delivery system of claim 1, wherein the skin care active comprises a negative logP active or a positive logP active.

3. The delivery system of claim 2, wherein the negative logP active comprises one or more of: niacinamide, kojic acid, 3-o-ethyl-L-ascorbic acid, zincidone (zinc salt of the L-pyrrolidone carboxylic acid), glucosamine, allantoin (5-ureidohydantoin, glyoxylic(acid) diureide), ascorbic acid 2-glucoside, ascorbic acid, lactic acid, glycolic acid, aspartic acid, threonine, glycine, L-ornithine, alanine, lysine, histidine, cysteine, valine, methionine, tyrosine, isoleucine, leucine, phenylalanine, tryptophan, hyaluronic acid, or sodium hyaluronate and the positive logP active comprises one or more of: caffeine, retinol, retinol derivatives, cholesterol, sterols, vitamin E and derivatives, benzophenone and chemical sunscreens, or salicylic acid.

4. The delivery system of claim 1, wherein the cosmetic composition comprises one or more of: a DNA repair enzyme, a sunscreen active, a humectant, a botanical extract, a peptide, an oil, a thickener, a surfactant, a vitamin, an antioxidant, a preservative, or a dermatologically acceptable carrier.

5. The delivery system of claim 1 wherein the nanocellulose comprises at least one of: cellulose nanofibers, cellulose nanocrystals, or bacterial nanocellulose.

6. The delivery system of claim 1 wherein the nanocellulose comprises phosphate-esterified cellulose nanofibers.

7. The delivery system of claim 1 wherein the nanocellulose comprises crystalline nanocellulose.

8. A method for regulating or improving topical delivery of a skin care active within the skin, the method comprising topically applying a composition comprising the skin care active and nanocellulose on the skin.

9. The method of claim 8, wherein the skin care active comprises one or more of: niacinamide, kojic acid, 3-o-ethyl-L-ascorbic acid, zincidone (zinc salt of the L-pyrrolidone carboxylic acid), glucosamine, allantoin (5-ureidohydantoin, glyoxylic(acid) diureide), ascorbic acid 2-glucoside, ascorbic acid, lactic acid, glycolic acid, aspartic acid, threonine, glycine, L-ornithine, alanine, lysine, histidine, cysteine, valine, methionine, tyrosine, isoleucine, leucine, phenylalanine, tryptophan, hyaluronic acid, or sodium hyaluronate.

10. The method of claim 8, wherein the skin care active comprises one or more of: one or more of caffeine, retinol or retinol derivatives, cholesterol, sterols, vitamin E and derivatives, benzophenone and chemical sunscreens, or salicylic acid.

11. The method of claim 8, wherein the nanocellulose comprises at least one of: cellulose nanofibers, cellulose nanocrystals, or bacterial nanocellulose.

12. A cosmetic or dermatologic composition comprising at least one skin care active, nanocellulose and cosmetically acceptable carriers, wherein the nanocellulose is present in amounts from about 0.001% to about 50% by the total weight of the composition.

13. The composition of claim 12, wherein the skin care active is a negative logP active or a positive logP active.

14. The composition of claim 13, wherein the negative logP active comprises one or more of: niacinamide, kojic acid, 3-o-ethyl-L-ascorbic acid, zincidone (zinc salt of the L-pyrrolidone carboxylic acid), glucosamine, allantoin (5-ureidohydantoin, glyoxylic(acid) diureide), ascorbic acid 2-glucoside, ascorbic acid, lactic acid, glycolic acid, aspartic acid, threonine, glycine, L-ornithine, alanine, lysine, histidine, cysteine, valine, methionine, tyrosine, isoleucine, leucine, phenylalanine, tryptophan, hyaluronic acid, or sodium hyaluronate.

15. The composition of claim 12, wherein the nanocellulose comprises at least one of: cellulose nanofibers, cellulose nanocrystals, or bacterial nanocellulose.

16. The composition of claim 12, wherein the nanocellulose comprises phosphate-esterified cellulose nanofibers.

17. A method of treating the appearance of human skin comprising administering to the skin an effective amount of the composition of claim 12.

18. The method of claim 17, wherein the treatment provides at least one cosmetic benefit selected from treating/preventing wrinkling, sagging, dry, aged and/or photodamaged skin, boosting, maintaining collagen levels in skin, boosting, maintaining decorin levels in skin, improving skin texture, smoothness or firmness and soothing irritated, red or sensitive skin.
